(19) United States
(12) Patent Application Publication
(10) Pub. No.: US 2022/0016350 A1
(43) Pub. Date: Jan. 20, 2022

(54) BACK FLOW PREVENTOR FOR A PREFILLED EMERGENCY SYRINGE AND LUER LOCK SYRINGE

(71) Applicants: Tyler J. ZAJAC, NOVI, MI (US); Charlie JORDAN, NOVI, MI (US); Alex RADDATZ, WATERFORD, MI (US); Lauren TROPPENS, SHELBY TOWNSHIP, MI (US)

(72) Inventors: Tyler J. ZAJAC, NOVI, MI (US); Charlie JORDAN, NOVI, MI (US); Alex RADDATZ, WATERFORD, MI (US); Lauren TROPPENS, SHELBY TOWNSHIP, MI (US)

(21) Appl. No.: 17/369,222

(22) Filed: Jul. 7, 2021

Related U.S. Application Data
(60) Provisional application No. 63/049,212, filed on Jul. 8, 2020.

(51) Int. Cl. A61M 5/315; A61M 5/28

(52) U.S. Cl. CPC A61M 5/31501; A61M 2005/3151; A61M 5/288; A61M 5/283

(57) ABSTRACT

A device to prevent prefilled syringe and disposable syringes from accidental separation and unwanted waste of medication. A backflow preventor allows either a prefilled medication vial or syringe plunger to pass through the clip provided as deformable protrusion or twist lock into a track which prevents the plunger or vial from separating from the syringe.

### TECHNICAL FIELD

The present invention relates to a prefilled emergency syringe or a disposable luer lock syringe and more particular to a luer adapter assembly for a prefilled emergency syringe of the type having an integral extending cannula and optionally hollow protective sheath.

### THE BACKGROUND OF THE INVENTION

Prefilled emergency syringes have become the standard for administering medications in the emergency and pre-hospital setting. The pre-dosing of medication allows for ease of use during urgent situations. The prefilled syringe reduces medication errors due to incorrect dosing caused by human error during stressful situations.

Emergency syringes that are used for drug administration at injection sites have either a needle or luer assembly for administration directly in-line with a patent IV line or saline lock. Common intravenous and intraosseous IV sites such as the antecubital vein or tibial plateau are a standard site used during emergency care.

During emergency care a prefilled syringe may be used in line with IV fluids. During an event such as a cardiac arrest, IV fluids will be forced into an IV site to support blood pressure. The pressure caused throughout the line can cause some complications with prefilled emergency syringes. When a luer lock syringe and a medication vial are assembled together, a rubber stopper threads into the inner threaded portion of a luer lock syringe. A major complication occurs when the pressure inside the prefilled vial builds up and forces the vial away from the rubber stopper. The rubber stopper and medication vial can separate thus wasting the medication.

In the pre-hospital setting, emergency providers have limited amount of resources to utilize during medical emergencies. Wasting medication due to an unexpected separation between a vial and the luer syringe can negatively impact patient outcomes.

Prefilled emergency syringes have been the leading method for emergency medication delivery in both hospital and pre-hospital settings. However, variations of products and limitations of availability make this invention ideal for all types of emergency syringes.

Due to increased national prefilled syringe shortages, many emergency and hospital settings are transitioning to disposable luer lock syringes and medication vials that must be drawn up and properly dosed.

In the hospital setting it is common practice to also use luer lock syringes for drawing up blood borne products (i.e. Blood, Cerebral Spinal Fluid, and Synovial Fluid). There is not usually a safe method in order to reduce cross contamination risk or exposure risk.

### DESCRIPTION OF THE INVENTION

This invention involves an alteration of standard syringes that can be utilized to help reduce medication waste as well as exposure to healthcare providers due to accidental dislocation or separation of syringes. Many critical situations induce stress to healthcare workers, especially in the event of administering critical care medications to urgent patients. In certain situations, a prehospital healthcare provider may have limited personnel to help with interventions. This design allows for the healthcare provider to multitask during emergency situations without worrying about syringe separation. This design can be implemented into current manufactured syringe designs.

In emergency prefilled syringes, the hollow syringe portion would have a back flow preventor clip, protrusion, track molded into the shaft of the syringe. The plastic or glass medication vial would have a lip at the proximal top of the vial, or another type of embodiment described within. The vial is introduced to the syringe via a rubber plunger which is threaded into the hollow syringe. The lip on the medication vial would depress the clip on the syringe. The clip would depress radially outward as the vial passes through it. The clip would return to standard position and would act as a backflow preventor. This design would prevent the separation of the medication vial from the syringe under pressure in emergency situations. Other embodiments described within show alternative designs and functions that do not include a clip that depresses radially outward. This design is ideal for all types of prefilled syringes and the designs can be integrated into current manufactured designs. This will help to solve a complication of prefilled medication syringes which will prevent loss of vital medication that critical patients require.

In disposable syringes, this same design can be implemented. During manufacturing of the syringe, the plunger is introduced into the shaft of the syringe. The plunger would pass through the clip and depress it radially outward. As the plunger advances forward, the clip would return to standard position and act as a safety preventing the plunger from separating from the syringe. Unlike the emergency prefilled syringe, these syringes come already together and require no assembling. This type of clip design would act as a safety when healthcare providers draw up bloodborne pathogens in the hospital and pre-hospital setting. Drawing up medication with a syringe is becoming more common and this design will prevent wasting medication due to accidental syringe/plunger dislocation.

The overall design of this clip can be placed in different locations along the shaft of the syringe. A clip could also be placed along the diameter of the syringe near the proximal opening. The exact type of clip and the number of clips included should be designed to maximize functionality and stability of both the syringe and vial. The design of the clip is not meant to be limiting. The design and function can be adjusted to meet specific design and recommendations of a manufacture to ensure ease of design. Renditions of this design herein are solely conceptual, actual design can be modified based on manufacture recommendations. This design can be incorporated into current marketed syringe vials and pre-filled medication vials.

### BRIEF DESCRIPTION OF DRAWINGS

FIGS. 1A-1B shows an exploded view of a prefilled emergency syringe in accordance with the exemplary embodiments described herein.

FIGS. 2A-2F shows the emergency syringe shaft with one example of embodiments in accordance to FIG. 1.

FIGS. 3A-3B shows the emergency syringe shaft with a secondary example of the embodiments in accordance to FIG. 1. FIG. 3C shows an end view of the emergency syringe shaft.

FIGS. 4A-4D shows the medication vial that threads into the shaft of the emergency prefilled syringe and the rubber stopper which seals the medication into the vial. FIG. 4E shows a close up portion of the side view of the medication vial.

FIGS. 5A-5C shows the assembly of the emergency prefilled syringe and the medication vial.

FIGS. 6A-6C shows the purpose of the backflow preventor clip as the medication vial threads into the syringe shaft in accordance to FIG. 1.

FIGS. 7A-7D shows a disposable syringe with one example of the embodiments described herein.

FIGS. 8A-8B shows a cross-section view of the disposable syringe of FIG. 7.

FIG. 9A shows a design of a disposable syringe plunger in accordance to FIG. 10.

FIGS. 10A-10B shows the assembly of a disposable syringe in accordance with the embodiments described herein.

FIG. 11 shows the use of the backflow preventor clip on a disposable syringe in accordance to FIG. 10.

FIGS. 12A-12E shows a different type of embodiment in accordance to FIG. 1.

FIGS. 13A-13D shows a reverse back flow preventor clip that would attach to the syringe vial in FIG. 1.

FIGS. 14A-14C shows a syringe via 3 with embodiments detailed in FIG. 13.

FIG. 15 shows a cross-sectional view of the assembly of FIGS. 13 and 14.

FIGS. 16A-16E shows a different type of embodiment in accordance with FIG. 1.

### DETAILED DESCRIPTION

Detailed, exemplary embodiments of the present invention are disclosed herein. However, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclose herein are not to be interpreted as limiting, but merely as a representative basis for teaching one skilled in the art to variously employ the present invention.

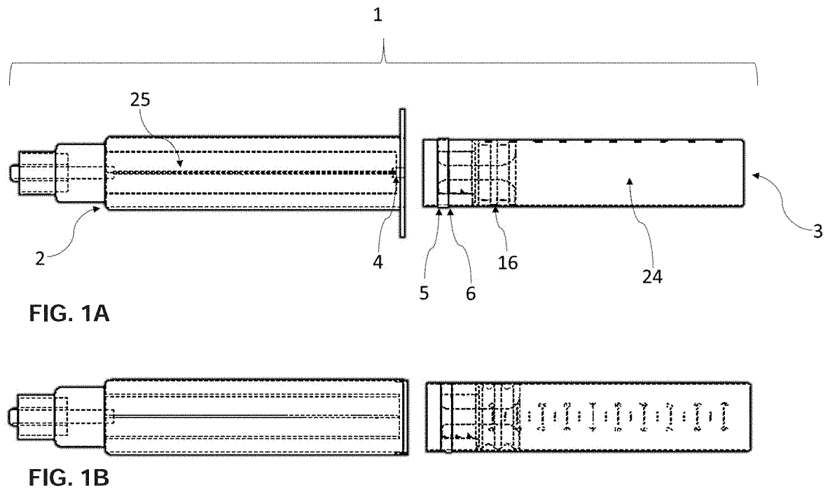

FIG. 1 shows a prefilled syringe assembly 1 in accordance with an exemplary embodiment described herein. This assembly 1 includes a syringe of a prefilled syringe 2 and a medication vial 3. Specific features of each of these components are described in more detail below in conjunction with associated drawing figures. The syringe portion 2 includes: a backflow preventor clip 4 which is molded directly into the convex shaft of the syringe. The medication vial 3 has an angled lip near the proximal tip 5 which matches the angle of the distal tip of the backflow preventor 4. On the medication vial 3 the proximal angle flares out and creates a lip 6. The rubber plunger 16 holds the medication 24 into the vial. The threads of the rubber plunger 16 thread into the shaft of the syringe 2 and is pierced by the needle 25. The vial 3 is pressed distally into the shaft of the syringe thus delivering the medication 24.

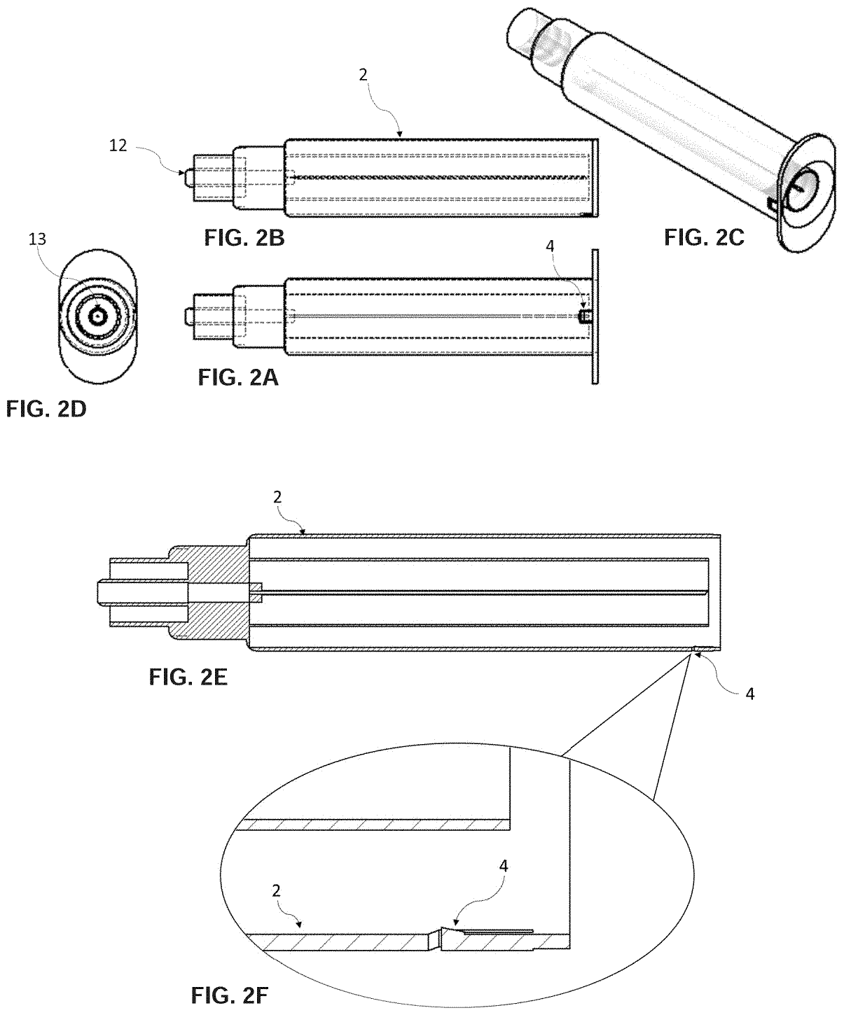

FIG. 2A shows a perspective view of the prefilled syringe shaft 2. The back flow preventor 4 is molded directly into the shaft of the syringe. The clip 4 depresses and allows the medication vial 3 to pass through it. At the distal tip of the syringe 2, a standard male luer lock tip 12 allows for connection to female luer lock connector.

FIG. 2B shows a lateral perspective of the prefilled syringe shaft 2. This view shows a secondary view of the backflow preventor clip 4.

FIG. 2C shows an isometric perspective of the prefilled syringe shaft 2.

FIG. 2D shows a perspective view of the posterior side of the syringe 2. A needle 13 embedded in the syringe 2 punctures the plunger located inside of the prefilled medication vial 3.

FIGS. 2E,2F shows a cross-sectional view of the syringe shaft 2. A close-up view of the syringe clip 4 shows the angled tip of the clip extending downwards into the shaft of the syringe.

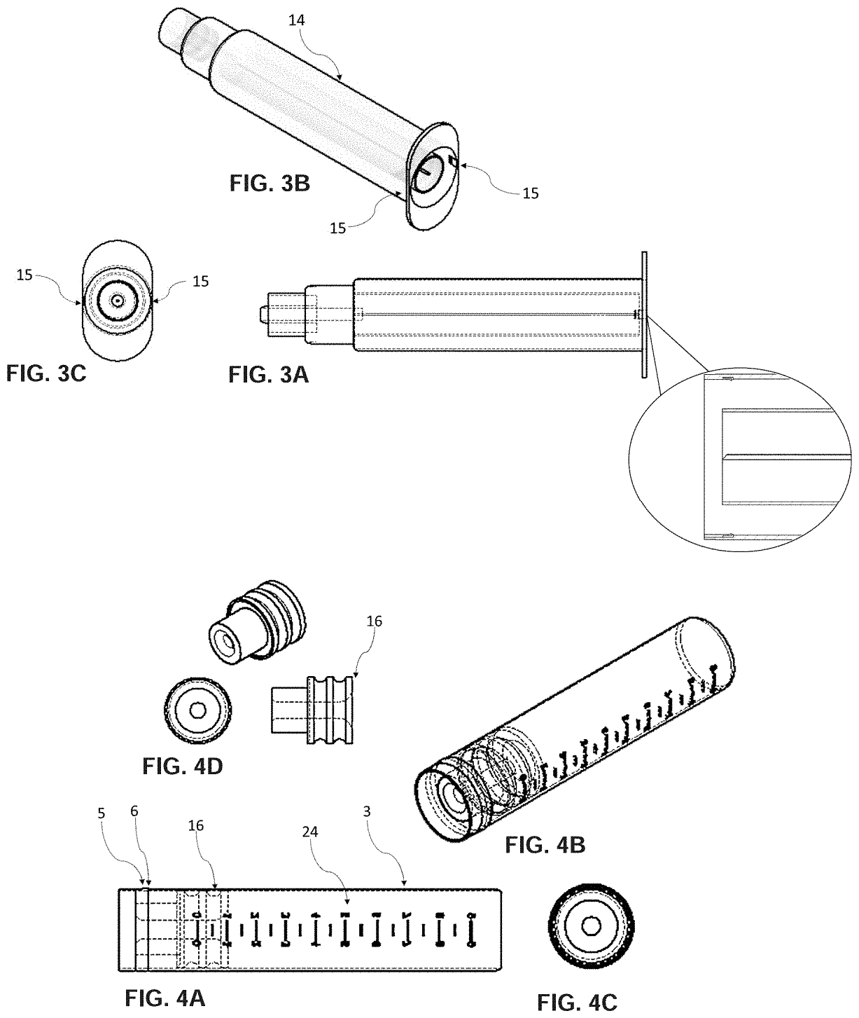

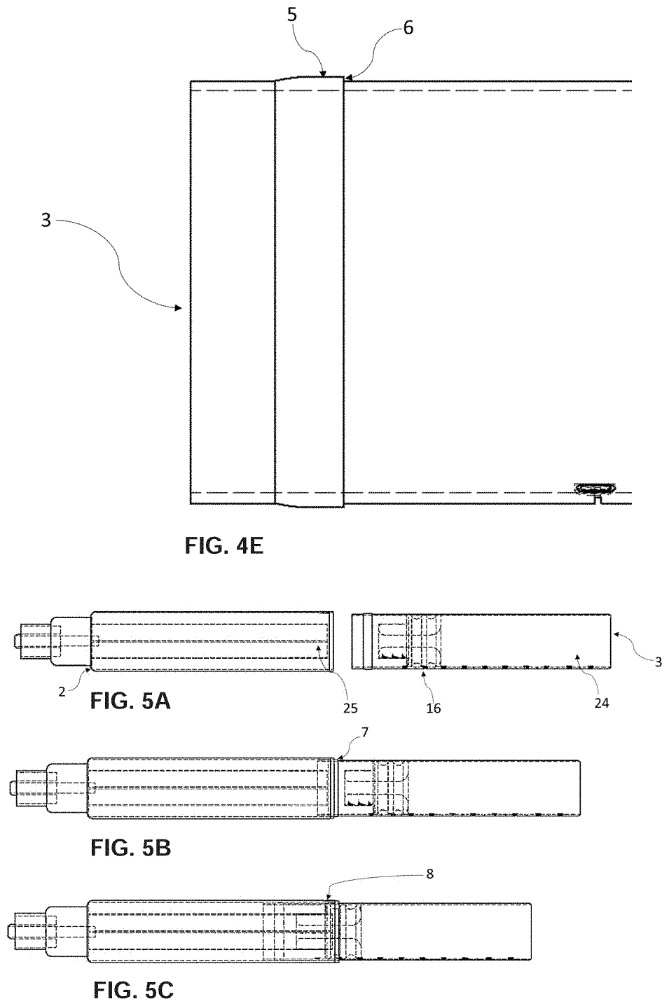

FIGS. 3A, 3B, and 3C respectively shows several perspective views of a prefilled syringe shaft 14. Syringe shaft 14 incorporates a secondary design of another type of backflow preventor clip design. The clip 15 is molded into the internal wall of the syringe shaft at the proximal end of the syringe. The clip 15 extends into the internal shaft of the syringe 14. The clip 15 would have a proximal angle that will allow for the FIG. 4 flared flange 5 to pass through and depress the clip.

FIGS. 4A, 4B, 4C, 4D and 4E respectively shows perspective views of the prefilled medication vial 3. The distal tip of the medication vial flanges out 5 which allows for direct contact with the backflow preventor clip 4, 15 as the vial is threaded into the syringe. The lip 6 catches on the distal side of the backflow preventor clip 4,15. The lip of the vial 6 bottoms out on the clip and prevents the vial from dislodging and separating from the syringe 2, 14. The medication vial 3 holds a prefilled dosage of medication, typically used in emergent situations. FIG. 4D represents the standard prefilled syringe plunger used in manufactured prefilled syringes. FIG. 4E is an exploded view of the lip 6 of the medication vial 3. The angle of the medication vial 5 depresses the clip 4 of the prefilled syringe.

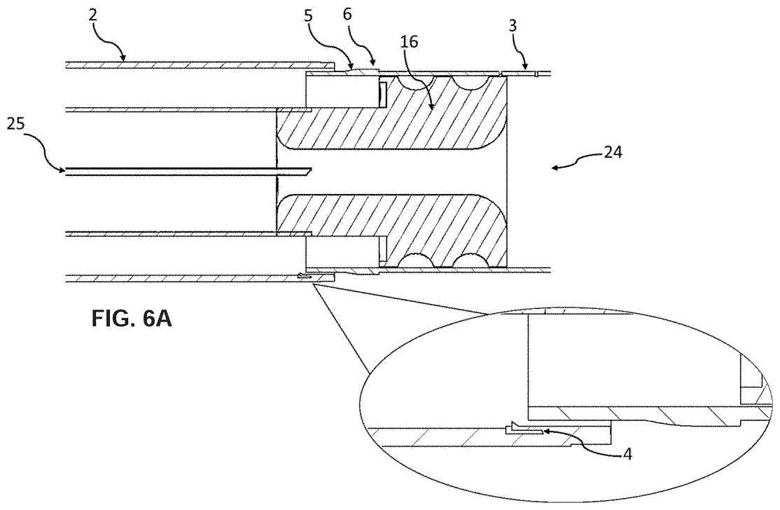

FIGS. 5A, 5B, and 5C respectively show a perspective view of the medication via 3 threading into the syringe 2. As the vial is screwed into the syringe at the distal end of the syringe 7. As the vial passes through the shaft of the syringe, the clip 8 is depressed radially outward. Once the vial passes the clip 8, the clip returns to standard position and will catch the lip 6 of the medication vial, thus preventing accidental dislocation of the of the syringe and vial. The rubber stopper 16 threads into a threaded portion of the syringe shaft 2. A needle 25 pierces the rubber stopper 16 and the medication 24 is delivered through the syringe.

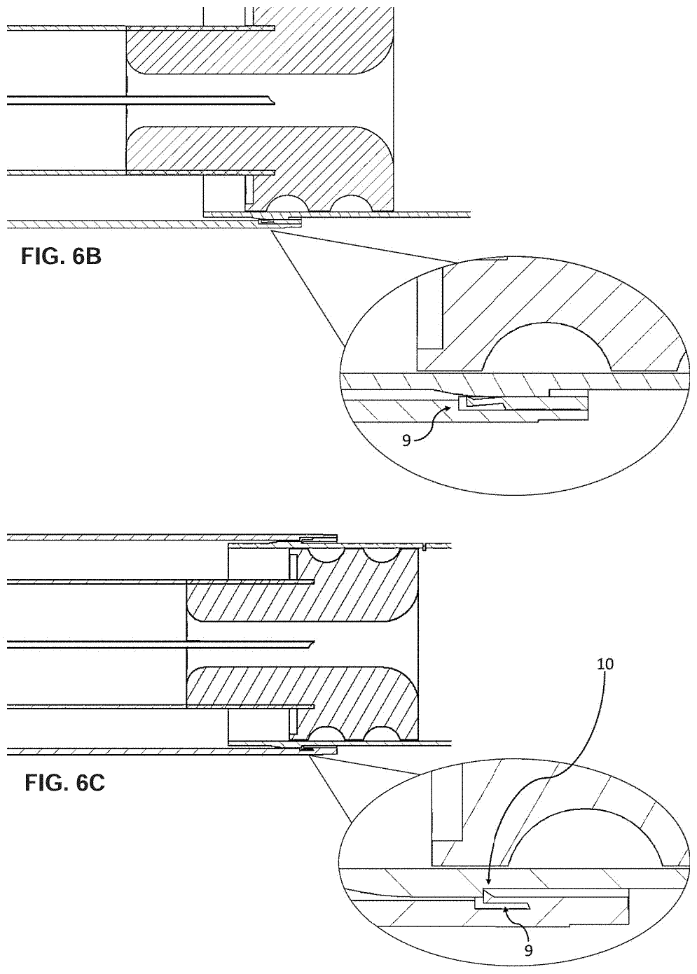

FIGS. 6A, 6B, and 6C shows an exemplary model of one example of the medication vial 3 passing through the clip 4 on the syringe shaft 2. As the vial enters the canal of the syringe, the needle 25 pierces the rubber stopper 16. As the vial 3 moves distally into the canal of the syringe, the flared portion of the vial 5 meets the backflow clip 4. As the vial 3 moves forward the angled portion of flanged vial 5 depresses the clip radially outward 9. Once the vial passes the clip 4 returns to standard position 11. As pressure builds up in the medication vial 3, the vial 3 begins to move backwards. When the medication vial 3 makes contacts with the clip 4, the clip 4 interacts and prevents the medication vial 3 from separating 10.

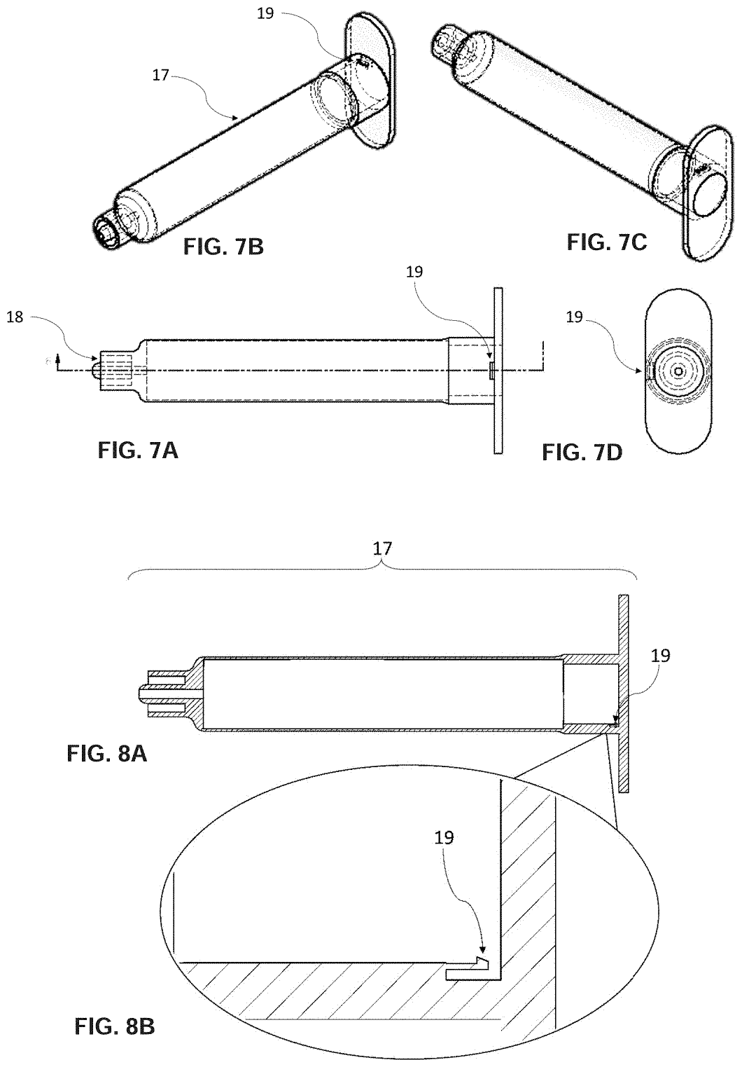

FIGS. 7A, 7B, 7C and 7D shows perspective views of a disposable syringe with an example of the embodiments explained herein. The syringe shaft 17 represents a standard disposable syringe. This embodiment can be perceived across different types of designs and sizes. At the distal tip of the syringe 18, a luer lock designed is portrayed in the figure. The luer lock 18 is designed to twist lock onto a clave lock. The backflow preventor clip 19 is molded into the syringe shaft.

FIGS. 8A, 8B represents a cross sectional view of FIG. 7. The backflow preventor clip 20 is one exemplary diagram of the embodiments discussed herein.

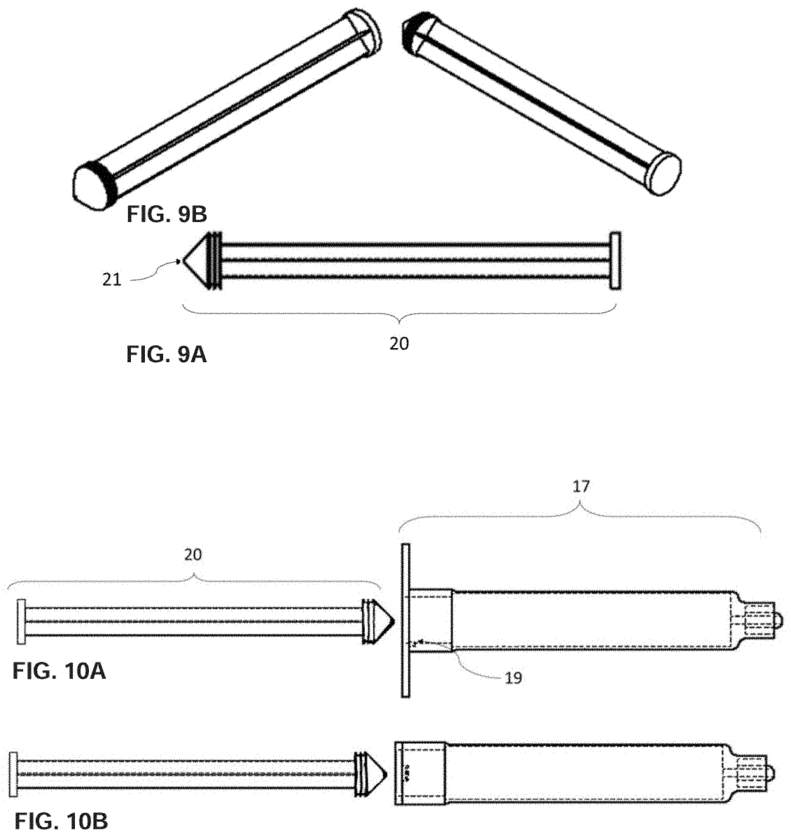

FIGS. 9A, 9B, and 9C shows a perspective view of a disposable syringe plunger. The syringe plunger 20 represents a standard manufactured syringe plunger for disposable syringes. A rubber cone shaped plunger 21 is attached to the distal tip of the plunger 20.

FIGS. 10A, 10B shows a perspective view of the complete assembly of a disposable syringe FIGS. 7 and 9. The distal end of the syringe plunger 20 is introduced into the proximal end of the syringe 17. As the plunger 20 passes through the backflow preventor clip 19, the clip locks and prevents the plunger from separating from the shaft of the plunger 17.

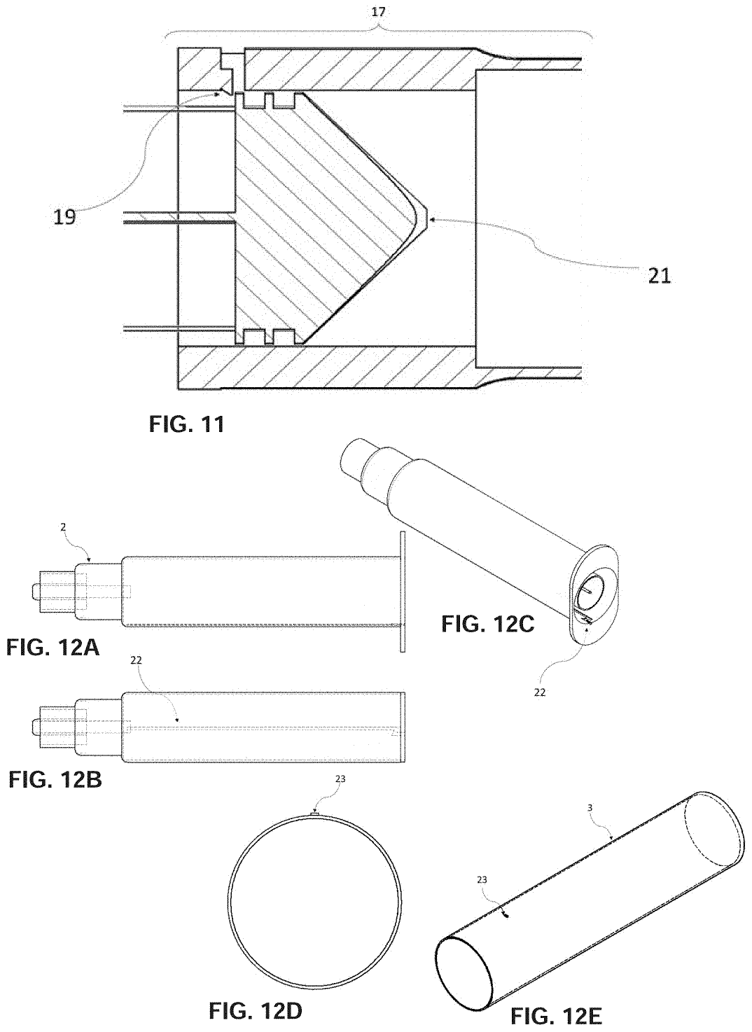

FIG. 11 shows a cross sectional view of FIG. 10 assembly. This cross-sectional view demonstrates how the back flow preventor clip 19 prevents the plunger tip 21 from separating from the shaft of the disposable syringe shaft 17.

FIGS. 12A and 12B shows a lateral view of the syringe 2 and a secondary option of the embodiments described above. A channel 22 can be used as a U-shape twist lock with an interior surface of the track that extends the length of the syringe 2.

FIG. 12C shows an isometric view that shows the channel 22 on the interior surface of the syringe 2.

FIGS. 12D and 12E shows the medication vial 3 and an outward protrusion 23, which would be inserted into the track 22.

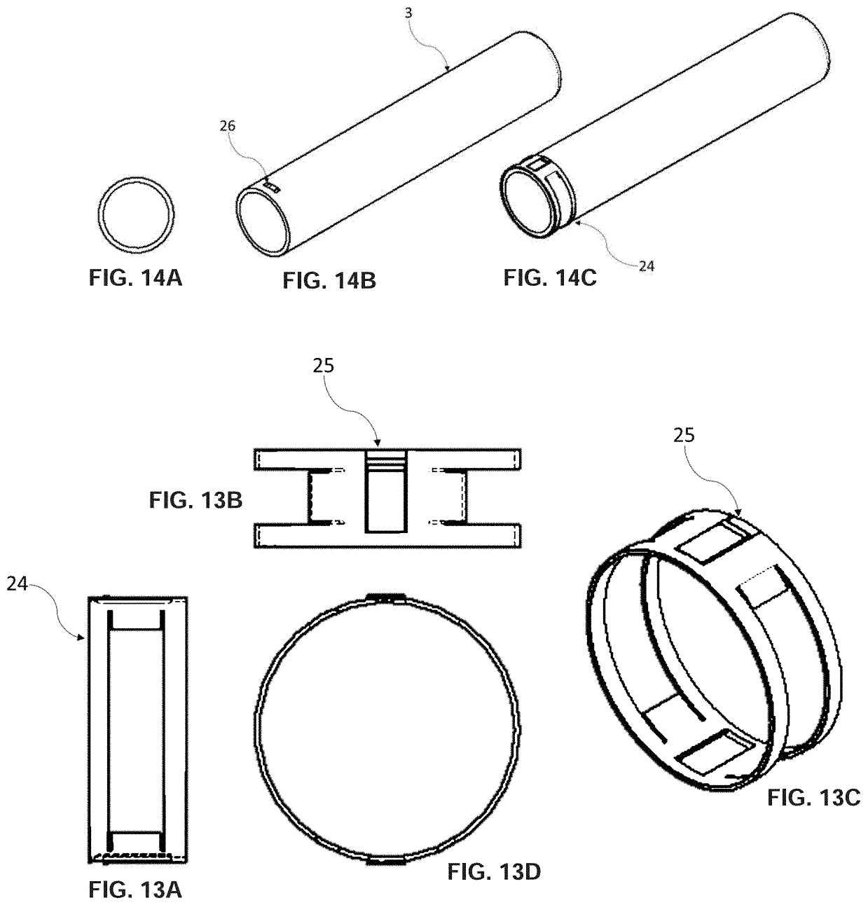

FIG. 13A, 13B, 13C, 13D shows a ring clip that is a reverse clip that would be placed on the medication vial 3.

FIG. 14 shows the medication vial 3 with both the clip 24 attached and unattached. The clip can be press fitted or similar attachment via grooves 26 in the vial 3. The clips 25 would depress radially inward towards the vial 3.

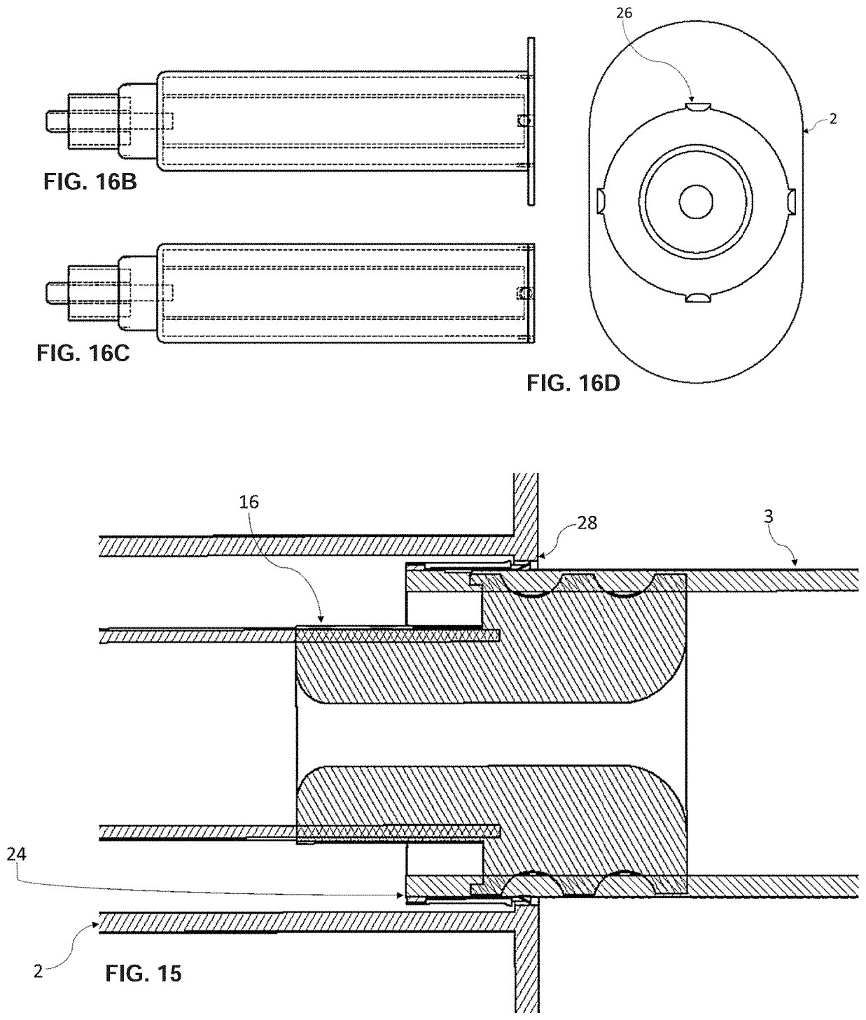

FIG. 15 is a cross-sectional view of the embodiments explained in FIGS. 13-14. A flange 28 extrudes medially towards the center of the syringe 2. The vial 3 and clip 24 is introduced into the syringe and clips 25 depress rotationally inward until the vial clears the flange. The clip 28 would return to standard position and the flange 28 would prevent separation.

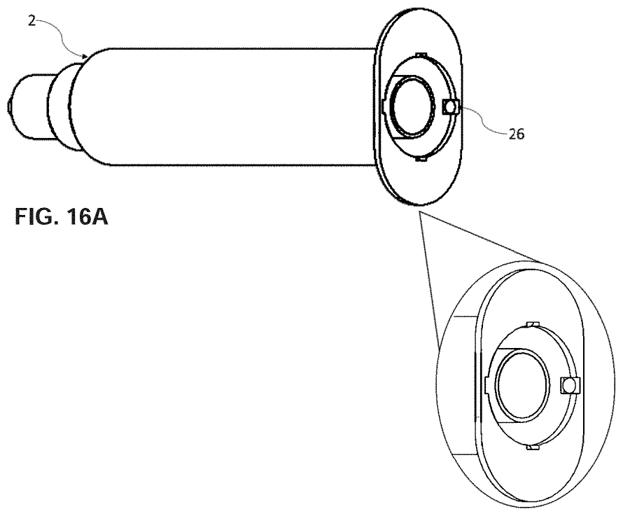

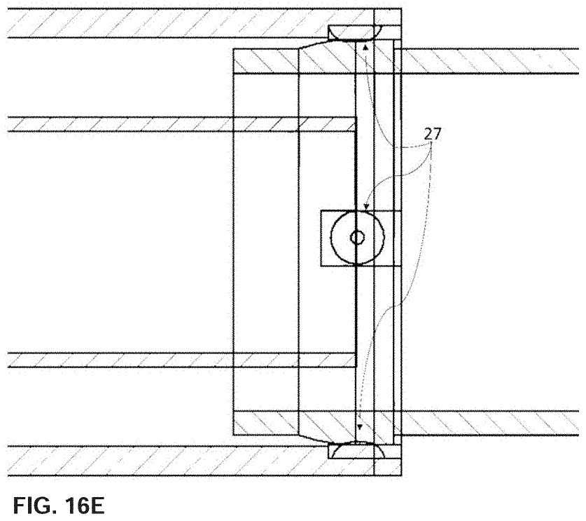

FIG. 16A, 16B, 16C, 16D shows another type of embodiment in reference to FIG. 1. The syringe portion 2 has a medial extrusion that will allow for resistance with the lip 6 of the medication vial 3. In this instance the flared flange 5 of the syringe vial 3 would push against a stationary extrusion 26 on the medial interior shaft of the syringe 2.

FIG. 16E shows a cross-sectional view of the FIG. 16 syringe assembled. As the vial 3 is inserted into the syringe 2, the flared flange 5 and lip of vial 6 will meet resistance with the medial shaft protrusion 26 and will then advance forward into the syringe. Back pressure will cause the lip 6 the hit the protrusion 26 and will cause the syringe to stop in place. Greater force will be required to separate the syringe 2 and vial 3.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined or substituted to form further embodiments of the invention. For example, the location and type of back flow preventor clip is meant not to be limiting to one specific design.

1. A syringe with a backflow preventor, comprising:
a syringe body having an open end and a second end longitudinally opposite from the open end;
a moving member configured to be inserted within the syringe body through the open end and movable longitudinally within the syringe body; and
a clip engaged between the syringe body and the moving member configured to prevent a separation of the syringe body and the moving member.

2. The syringe according to claim 1, wherein the clip is disposed on the syringe body proximate to the open end of the syringe body.

3. The syringe according to claim 1,
wherein the moving member moves within the syringe body in a first direction extending longitudinally from the open end to the second end and a second direction longitudinally from the second end to the open end, and
wherein the clip limits an extent of movement of the moving member in the second direction.

4. The syringe according to claim 1,
wherein the clip has a sliding surface and a locking surface, and
wherein the moving member has an engagement surface configured to slide against the sliding surface and lock against the locking surface.

5. The syringe according to claim 1,
wherein the syringe body has an internal circumferential surface and a recessed portion extending radially outward from the internal circumferential surface, and
wherein in the clip is flexibly engaged between the syringe body and the moving member and configured to flex between an insertion position in which the clip is disposed within the recessed portion and a locking position in which at least a portion of the clip extends radially inward of the internal circumferential surface of the syringe body.

6. The syringe according to claim 1,
wherein in the moving member is one of a shaft of a disposable syringe and a prefilled medication vial.
